(19) United States
(12) Patent Application Publication
(10) Pub. No.: US 2004/0005342 A1
(43) Pub. Date: Jan. 8, 2004

(54) Topical administration of ascorbic acid to reinforce the cohesion of the dermo-epidermal junction

(75) Inventor: Francoise Bernerd, Paris (FR)

Correspondence Address: BURNS, DOANE, SWECKER & MATHIS, L.L.P., P.O. BOX 1404, Alexandria, VA, 22313-1404, US

(73) Assignee: SOCIETE L'OREAL S.A., Paris (FR)

(21) Appl. No.: 10/358,888

(22) Filed: Feb. 6, 2003

Related U.S. Application Data
(60) Provisional application No. 60/356,171, filed on Feb. 14, 2002.

(30) Foreign Application Priority Data
Feb. 7, 2002 (FR) 02/01510

(51) Int. Cl. A61K 7/00; A61K 31/375

(52) U.S. Cl. 424/401; 514/474

(57) ABSTRACT

The present invention relates to the pharmaceutical or cosmetic use of a composition comprising, in a pharmaceutically or cosmetically acceptable medium, ascorbic acid or an analogue thereof to reinforce the cohesion of the dermo-epidermal junction.

The invention also relates to the pharmaceutical or cosmetic use of a composition comprising, in a pharmaceutically or cosmetically acceptable medium, ascorbic acid or an analogue thereof to increase the synthesis of tenascin and/or collagen VII.

The invention also relates to the use of ascorbic acid or of an analogue thereof to reinforce the cohesion of the dermo-epidermal junction of reconstructed skins.

### CROSS-REFERENCE TO PRIORITY/PROVISIONAL APPLICATIONS

This application claims priority under 35 U.S.C. § 119 of FR-02/01510, filed Feb. 7, 2002, and of provisional application Serial No. 60/356,171, filed Feb. 14, 2002, both hereby expressly incorporated by reference.

### BACKGROUND OF THE INVENTION

1. Technical Field of the Invention

The present invention relates to the pharmaceutical or cosmetic use, or regime or regimen which comprises administration of a composition comprising, in a pharmaceutically or cosmetically acceptable medium, ascorbic acid or an analogue thereof to reinforce the cohesion of the dermo-epidermal junction.

2. Description of the Prior Art

Ascorbic acid (or vitamin C) is commonly used in topical compositions since it is known that it has a beneficial effect on the skin. In particular, it stimulates the synthesis of connective tissue, reinforces the defences of skin tissue against external attacking factors such as ultraviolet radiation and pollution, compensates for vitamin E deficiency of the skin, depigments the skin and has a free-radical-scavenging function.

Another activity of vitamin C described in the literature is its action on fibroblasts. Studies have shown that vitamin C makes it possible to increase the production of type I and III collagen, with action on different levels of regulation. Vitamin C especially has post-translational activity, by interacting with the enzymes for collagen I and III maturation, as a co-factor for prolylhydroxylase.

Recent studies have also related to the effect of vitamin C on transcription. Vitamin C thus regulates the transcription of the collagen I gene (Slack J L, Liska D J, Bornstein P. Regulation of the expression of the type I collagen genes. Am. J. Med. Genet. 1993, 45(2): 140-51) and of the collagen III mRNAs in skin fibroblasts (Geesin J C, Darr D, Kaufman R, Murad S, Pinnell S R. Ascorbic acid specifically increases type I and type III procollagen mesRNA levels in human skin fibroblast. J. Invest. Dermatol. 1988, 90(4): 420-4). In contrast, it appears that ascorbic acid has little effect on transcription of the type IV collagen gene (Tajima and Pinnell, 1996). Finally, more recent studies have shown that the addition of vitamin C to reconstructed epidermal cultures had an influence on the barrier function by improving the lipid profile of the epidermal cells (Ponec M, Weerheim A, Kempenaar J, Mulder A, Gooris G S, Bouwstra J, Mommaas A M. The formation of competent barrier lipids in reconstructed human epidermis requires the presence of vitamin C. J. Invest. Dermatol. 1997, 109(3): 348-55).

Human skin consists of two tissues, one a surface tissue, the epidermis, and the other a deep tissue, the dermis.

Natural human epidermis is composed mainly of three types of cells, namely keratinocytes, which form the vast majority, melanocytes and Langerhans cells. Each of these three types of cells contributes, via its intrinsic functions, in the essential role played in the body by the skin, especially the role of protecting the body against external attacking factors (the climate, ultraviolet rays, tobacco, etc.), which is also known as the “barrier function”.

The dermis provides the epidermis with a solid support. It is also its nourishing element. It consists mainly of fibroblasts and of an extracellular matrix composed mainly of collagen, elastin and a substance known as ground substance. These components are synthesized by the fibroblasts. Leucocytes, mastocytes and tissue macrophages are also found therein. Finally, the dermis is interlaced with blood vessels and nerve fibers.

The cohesion between the epidermis and the dermis is provided by the dermo-epidermal junction. This is a complex region about 100 nm thick, which comprises the basal pole of the basal keratinocytes, the epidermal membrane and the sub-basal zone of the superficial dermis (Bernard P. Structure de la jonction dermo-épidermique. Objectif peau. [Structure of the dermo-epidermal junction. Objective: skin.] 2001, 68: 87-93).

In structural terms, hemidesmosomes, in which are inserted keratin filaments (hemidesmosome-tonofilament complex), are distributed over the plasma membrane of the basal keratinocytes.

As regards the hemidesmosome-tonofilament complexes, there are anchoring filaments that pass through the epidermal basal membrane. The anchoring filaments are connected to the epidermal side of laminin 5.

Finally, anchoring fibrils constitute the sub-basal network. These are curvilinear structures that arise and terminate on the deep face of the basal membrane and into which are inserted collagen I, III and V fibers. It has been shown that these anchoring fibrils, which may be clearly visualized by electron microscopy, are composed of type VII collagen. Type VII collagen is synthesized by the keratinocytes and the fibroblasts, but mainly by the keratinocytes (Aumailley M, Rousselle P. laminins of the dermo-epidermal junction. Matrix Biology, 1999, 18: 19-28; Nievers M, Schaapveld R, Sonnenberg A. Biology and function of hemidesmosomes. Matrix Biology, 1999, 18: 5-17).

Collagens are major proteins of the extracellular matrices. To date, 20 types of collagen have been identified and named from I to XX. Different families are distinguished among these types depending on the structures formed:

the family of fibrillar collagens (types I, II, III, V and XI) which form fibers;

the family of collagens forming the network of the basal membranes, which comprises type IV and type XVII collagen;

collagens forming hexagonal networks (type VIII and type X), pearled filaments (type VI), and FACITs (types IX, XII, XIV, XVI, XIX and XX);

the anchoring fibrils, which correspond to type VII;

multiplexins (types XV and XVIII) and

collagen of type XIII, the precise functions of which are not known at the present time.

In the skin, the collagens mainly present throughout the dermis are the type I and III collagens which form the extracellular matrix of the entire dermis (these collagens constitute 70-80% of the dry weight of the dermis).

Collagens represent a family of very wide diversity; specifically, each type of collagen and the chains of which it is made are derived from different genes. For example, the type I collagens are composed of two chains COL1A1 and COL1A2, the genes for which are located on chromosomes 17q and 7q, respectively. The type VII collagen is composed of a single chain and its gene is located on chromosome 3p. Moreover, collagens are not all synthesized by the same cell type; type I and III collagens are essentially produced by the dermal fibroblasts, whereas type VII collagen is produced by the epidermal keratinocytes. Finally, the regulation of their expression differs from one collagen to another, for example collagens I and VII are not regulated in the same way by certain cytokines; specifically, TNF-α and leukoregulin stimulate collagen VII and negatively regulate collagen I.

The cohesion between the two tissues, epidermis and dermis, is essential for the integrity of the skin as a whole and allows resistance to external attacking factors of mechanical type (friction, blows, etc.).

The dermo-epidermal junction is also a structure that conditions the state of the surface of the skin.

Thus, a dermo-epidermal junction with integral anchoring structures is now folded, thus making it possible to increase the area of the zone of contact between the dermis and the epidermis, to promote the exchanges between these two tissues, to reinforce their cohesion and to improve the appearance of the epidermis.

In cases where the anchoring structures are impaired (deficiency of collagen VII or tenascin synthesis, aging, etc.), this causes flattening of the dermo-epidermal junction. The exchanges are reduced, the two tissues are less solidly attached, the epidermis folds and, since the skin is less firm and less taut, wrinkles appear and the skin becomes more susceptible to mechanical attack.

The absence or non-functionality of the compounds of the dermo-epidermal junction causes pathologies of epidermolysis bullosa type. The cleavage zone is determined by the location of the deficient protein.

This is especially the case for individuals with mutations located in the gene coding for collagen VII. These mutations induce epidermolysis bullosae (EB), such as the Hallopeau-Siemens type of recessive dystrophic EB, mild-type of recessive dystrophic EB and dominant dystrophic EB. These genetic diseases are characterized by the absence of formation of these anchoring fibrils, the result of which is extreme fragility of the skin with detachment of the two tissues (dermis-epidermis), resulting in the formation of “bullae”. Other types of epidermolysis bullosa may also have an auto-immune origin, with production of auto-antibodies directed against collagen VII, for instance acquired epidermolysis bullosa, bullous systemic lupus erythematosus and bowel disease. In all cases, epidermolysis bullosae give rise to anomalies that are very incapacitating since the afflicted individuals must be extremely prudent and must not provoke any tension or trauma that might result in a mechanical effect on the dermo-epidermal junction.

Akiyama et al. have moreover described that collagen VII also plays an important role in human hair follicles, especially in the basal membranes of the matrix (peripapillary zone) and in the bulge membrane (bulge of the top part of the bulb). These two zones contain cells with strong mitotic potential, in particular keratinocytes that give rise to the hair stem. These authors have indicated that the collagen VII present in the follicle is essential for the expression of this mitotic activity (Akiyama M, Dale B A, Sun T T, Holbrook K A. Characterisation of hair follicle bulge in human fetal skin: the human fetal bulge is a pool of undifferentiated keratinocytes. J. Invest. Dermatol. 1995, 105: 844-50).

Another major constituent of the dermo-epidermal junction is tenascin. Tenascin is a glycoprotein of the extracellular matrix, also known as tenascin-C. It essential function lies in epithelium-mesenchyma interactions especially during embryogenesis.

In the skin, tenascin is found at the sub-epidermal level in the papillary dermis, but also around the blood vessels and related structures. Its expression is greatly increased in situations of hyperproliferation, for instance psoriasis and tumours, but also in cicatrization. Tenascin is produced in the skin by the two major cell types, the keratinocytes and the fibroblasts. One of its functions lies in cellular adhesion. Specifically, the strong positive regulation of tenascin in migrating keratinocytes during cicatrization strongly suggests an essential role of adhesion of the keratinocytes to the connective tissue, thus ensuring good dermo-epidermal cohesion (Crossin K L. Tenascin: a multifunctional extracellular matrix protein with a restricted distribution in development and disease. J. Cell. Biochem. 1996, 61: 592-598; Latijnhouwers M, Bergers M, Ponec M, Dijkman H, Andriessen M, Schlkwijk J. Human epidermal keratinocytes are a source of tenascin-C during wound healing. J. Invest Dermatol., 1997, 108: 776-783; Steijlen P M, Maessen E, Kresse H, Van Vlijmen I M J J, Verstraeten A A, Traupe H, Schalkwijk J. Expression of tenascin, biglycan and decorin in disorders of keratinization, Br. J. Dermatol., 1994, 130: 564-568).

### SUMMARY OF THE INVENTION

The Applicant has demonstrated, surprisingly, that ascorbic acid (or vitamin C) makes it possible to increase the synthesis of tenascin and collagen VII.

Vitamin C may thus be used in pharmaceutical compositions, in particular dermatological compositions, or cosmetic compositions, to reinforce the cohesion of the dermo-epidermal junction and/or to reinforce the expression of the mitotic potential of the hair follicles of the scalp.

To the Applicant's knowledge, only certain retinoids, especially retinoic acid or retinol (Woodley D T, Zelickson As, Briggaman R A, Hamilton T A, Weiss J S, Ellis C N, Voorhees J J. Treatment of photoaged skin with topical tretinoin increases epidermal anchoring fibrils. A preliminary report. JAMA. 1990, 263(22): 3057-9), extracts of the plant Potentilla erecta (U.S. Pat. No. 6,193,975) and extracts of the yeast Saccharomyces cerevisiae (yeast extract sold under the trademark Toniskin by Silab) make it possible at the present time to obtain the same effects. The irritant effect of retinoids on the skin is moreover known.

Patent application EP-0,738,508 describes a composition comprising a saccharide ester of ascorbic acid and a sulphonic UVA screening agent, which is useful for combatting pigmentation marks and aging; vitamin C is described therein as an antioxidant and a free-radical scavenger. Patent application WO 02/15860 describes an antioxidant topical composition comprising vitamin C, described as an antioxidant and a free-radical-scavenging compound that promotes the action of vitamin E and stimulates the synthesis of collagen I. The subject of these two documents is to propose compositions in which the ascorbic acid shows good stability.

No prior art document describes the beneficial effect of vitamin C on the dermo-epidermal junction, whether by increasing the synthesis of tenascin or by increasing the synthesis of collagen VII.

One subject of the present invention is thus the cosmetic use of a composition comprising, in a cosmetically acceptable medium, ascorbic acid or an analogue thereof to reinforce the cohesion of the dermo-epidermal junction.

A subject of the invention is also the cosmetic use of a composition comprising, in a cosmetically acceptable medium, ascorbic acid or an analogue thereof for increasing the synthesis of tenascin.

Another subject of the invention is the cosmetic use of a composition comprising, in a cosmetically acceptable medium, ascorbic acid or an analogue thereof for increasing the synthesis of collagen VII.

### DETAILED DESCRIPTION OF BEST MODE AND SPECIFIC/PREFERRED EMBODIMENTS OF THE INVENTION

By virtue of the properties of these proteins, collagen VII and tenascin, which participate in the attachment and anchoring of the epidermal keratinocytes in the dermal extracellular matrix, dermo-epidermal cohesion as a whole is thus ensured. Furthermore, their involvement in the mechanical aspect increases the resistance of the skin to mechanical stress, rubbing and friction. This aspect is especially illustrated by the formation of bullae or blisters in the diseases (genetic or auto-immune, mentioned above) associated with a deficiency of collagen VII. More commonly, this aspect is also illustrated by skin that shows deficiencies in the dermo-epidermal junction and that is less firm and elastic.

It is has also been shown that both tenascin and collagen VII are decreased or negatively regulated by the glucocorticoids that are known to induce fragility of the skin. Increasing collagen VII thus make it possible to counteract this fragility. It has also been mentioned that atrophy of the skin occurs in diseases associated with a collagen VII deficiency.

These proteins, collagen VII and tenascin, of the dermo-epidermal junction also have functions as mediators of cellular adhesion, in particular for epidermal cells, as mediators for the migration of keratinocytes on the connective matrix, and participate in the transmission of signals from the extracellular matrix to the epidermis (Burgeson R E and Christiano A M. Current Opinion in cell Biology, 1997, 9: 651-658).

The dermo-epidermal junction thus plays a role as a dynamic interface between the connective tissue and the epithelium. It governs the structural integrity of the skin organ in its entirety, and controls the passage of molecules between the dermis and the epidermis.

This zone is essential during skin cicatrization or remodelling processes.

Thus, the cosmetic composition will be intended in particular for reinforcing the anchoring structures between the dermis and the epidermis and/or for improving the binding and adhesion of the epidermal cells to the matrix support and/or for improving the biomechanical properties of the skin and/or for increasing the resistance of the skin to mechanical attacking factors such as rubbing, tension and friction and/or for promoting skin repair.

In one particular variant, the invention relates to the cosmetic use of ascorbic acid or an analogue thereof for any application in which it is desired to increase the synthesis of collagen VII.

Thus, the cosmetic composition according to the invention may be intended for improving the condition of the scalp, for improving the growth of good-quality hair, and for retarding or slowing down hair loss.

In this respect, the cosmetic composition may be a hair care lotion, such as a lotion for preventing hair loss.

The cosmetic compositions according to the present invention may contain an effective amount of ascorbic acid or analogues thereof, i.e., the amount required to obtain the expected effects according to the invention. To give an order of magnitude, this amount preferentially represents from 0.001% to 20% relative to the total weight of the composition, preferably from 0.1% to 15% relative to the total weight of the composition and advantageously from 1% to 10% relative to the total weight of the composition.

In addition, the composition of the invention will also be used for a time that is sufficient to obtain the expected effects according to the invention. To give an order of magnitude, this duration may be at least 15 days, but it may also be more than 4 weeks or even more than 8 weeks.

The invention also relates to the use of ascorbic acid or an analogue thereof for the preparation of a pharmaceutical composition, especially a dermatological composition, for treating disorders associated with a deficiency of cohesion between the dermis and the epidermis, in particular those associated with weakening of the dermo-epidermal junction.

Another aspect of the invention is the use of ascorbic acid or an analogue thereof in the preparation of a pharmaceutical composition, especially a dermatological composition, for treating disorders associated with an insufficiency of tenascin synthesis.

In another aspect of the invention, a subject of the invention is the use of ascorbic acid or an analogue thereof in the preparation of a pharmaceutical composition, especially a dermatological composition, for treating disorders associated with an insufficiency of collagen VII synthesis.

By way of example, these disorders may be alopecia associated with collagen formation disorders.

The pharmaceutical compositions according to the invention may more particularly be dermatological compositions for promoting skin cicatrization, in particular in the treatment of wounds, optionally in addition to local therapeutic treatments, especially for improving the quality of the skin during and after cicatrization.

To give an order of magnitude, this amount preferably represents from 0.001% to 20% relative to the total weight of the composition, more preferably from 0.1% to 15% relative to the total weight of the composition and advantageously from 1% to 10% relative to the total weight of the composition.

For the preparation of the cosmetic and pharmaceutical compositions of the invention, ascorbic acid may be used, which is generally in L form since it is usually extracted from natural products, or analogues thereof.

On account of its chemical structure (a-keto lactone) which makes it very sensitive to certain environmental parameters such as light, heat and aqueous media, it may be advantageous to use the ascorbic acid in the form of a saccharide ester of ascorbic acid or a metal salt of phosphoryl ascorbic acid.

The saccharide esters of ascorbic acid that may be used in the invention are especially the glucosyl, mannosyl, fructosyl, fucosyl, galactosyl, N-acetylglucosamine and N-acetylmuramic derivatives of ascorbic acid, and mixtures thereof, and more especially ascorbyl-2 glucoside or 2-O-α-D-glucopyranosyl L-ascorbic acid or 6-O-β-D-galactopyranosyl L-ascorbic acid. The latter compounds and processes for preparing them are described in particular in documents EP-A-487,404, EP-A-425,066 and J 05 213 736.

As regards the metal salt of phosphoryl ascorbic acid, it may be chosen from alkali metal ascorbyl phosphates, alkaline-earth metal ascorbyl phosphates and transition metal ascorbyl phosphates.

The ascorbic acid analogues are, more particularly, its salts, such as, especially, sodium ascorbate, magnesium or sodium ascorbyl phosphate, its esters, such as, especially, its acetic, propionic or palmitic esters, or its sugars, such as, especially, glycosyl ascorbic acid.

The pharmaceutical and cosmetic compositions according to the invention may be in any form that is suitable for topical application, especially in the form of aqueous, aqueous-alcoholic or oily solutions, dispersions of the lotion or serum type, aqueous, anhydrous or oily gels, emulsions of liquid or semi-liquid consistency of the milk type, obtained by dispersing a fatty phase in an aqueous phase (O/W) or conversely (W/O), or a triple emulsion (W/O/W or O/W/O), suspensions or emulsions of soft, semi-solid or solid consistency of the cream or gel type, microemulsions, or alternatively microcapsules, microparticles or vesicular dispersions of ionic and/or nonionic type. These compositions are prepared according to the usual methods well known to those skilled in the art.

According to one particular embodiment of the invention, the compositions according to the invention are in the form of a support and a separate solid, which are intended to be mixed together extemporaneously, the solid consisting essentially of pulverulent ascorbic acid. Such an arrangement is described, for example, in patent application FR-94/03982 filed by the Applicant, and Example 1 of the present patent application illustrates this type of formulation.

In a known manner, the pharmaceutical and cosmetic compositions of the invention may also contain adjuvants that are common in the fields under consideration, such as hydrophilic or lipophilic gelling agents, anti-glycation agents, agents for stimulating the synthesis of dermal or epidermal macromolecules and/or for preventing their degradation, agents for stimulating the proliferation of fibroblasts or keratinocytes and/or for differentiating keratinocytes, tensioning agents, lipolytic active agents or active agents that have a favourable, direct or indirect activity on reducing adipose tissue, agents that act on the capillary circulation, hydrophilic or lipophilic active agents, preserving agents, antioxidants, solvents, fragrances, fillers, screening agents, pigments, odor absorbers and dyestuffs. The amounts of these various adjuvants are those conventionally used in the field under consideration, and, for example, from 0.01% to 20% relative to the total weight of the composition. Depending on their nature, these adjuvants may be introduced into the fatty phase, into the aqueous phase, into the lipid vesicles and/or into the nanoparticles. In any case, these adjuvants and the proportions thereof will be chosen so as not to harm the properties desired according to the invention.

The compositions of the invention may thus also comprise hydrophilic or lipophilic active agents, and especially active agents capable of complementing the effect of ascorbic acid.

The term “anti-glycation agent” means a compound for preventing and/or reducing the glycation of skin proteins, in particular of dermal proteins such as collagen.

Examples of anti-glycation agents are plant extracts of the Ericacea family, such as an extract of blueberry (Vaccinium angustifolium); ergothioneine and its derivatives; and hydroxystilbenes and their derivatives, such as resveratrol and 3,3′,5,5′-tetrahydroxystilbene. These anti-glycation agents are described in patent applications FR 99/16166, FR 00/08158, FR 99/09267 and FR 99/16168, respectively. Resveratrol is particularly preferred for use in this invention.

Among the active agents for stimulating dermal macromolecules, mention may be made of those that act:

either on collagen synthesis, such as extracts of Centella asiatica; asiaticosides and derivatives; synthetic peptides such as lamin, biopeptide CL or the palmitoyloligopeptide sold by Sederma; peptides extracted from plants, such as the soybean hydrolysate sold by Coletica under the trademark Phytokine®; plant hormones such as auxins;

or on elastin synthesis, such as the extract of Saccharomyces cerivisiae sold by LSN under the trademark Cytovitin®; and the extract of the alga Macrocystis pyrifera sold by SECMA under the trademark Kelpadelie®;

or on glycosaminoglycan synthesis, such as the product of fermentation of milk with Lactobacillus vulgaris, sold by Brooks under the trademark Biomin yogourth®; the extract of the brown alga Padina pavonica sold by Alban Müller under the trademark HSP3®; and the extract of Saccharomyces cerevisiae available especially from the company Silab under the trademark Firmalift® or from the company LSN under the trademark Cytovitin®;

or on fibronectin synthesis, such as the extract of the zooplankton Salina sold by Seporga under the trademark GP4G®; the yeast extract available especially from the company Alban Müller under the trademark Drieline®; and the palmitoyl pentapeptide sold by Sederma under the trademark Matrixil®;

or on metalloprotease (MMP) inhibition, such as, more particularly, MMP 1, 2, 3 or 9. Mention may be made of: retinoids and derivatives, isoflavonoids, oligopeptides and lipopeptides, lipoamino acids, the malt extract sold by Coletica under the trademark Collalift®; extracts of blueberry or of rosemary; lycopene; isoflavones, their derivatives or plant extracts containing them, in particular extracts of soybean (sold, for example, by Ichimaru Pharcos under the trademark Flavosterone SB®), of red clover, of flax, of kakkon or of sage;

or on the inhibition of serine proteases such as leukocyte elastase or cathepsin G. Mention may be made of: the peptide extract of Leguminosa seeds (Pisum sativum) sold by LSN under the trademark Parelasty®; and heparinoids and pseudodipeptides.

Among the active agents that stimulate epidermal macromolecules, such as fillagrin and keratins, mention may be made especially of the extract of lupin sold by Silab under the trademark Structurine®; the extract of beech Fagus sylvatica buds sold by Gattefosse under the trademark Gatuline®; and the extract of the zooplankton Salina sold by Seporga under the trademark GP4G®.

The agents for stimulating the proliferation of fibroblasts that may be used in the composition according to the invention may be chosen, for example, from plant proteins or polypeptides, extracts, especially of soybean (for example an extract of soybean sold by LSN under the name Eleseryl SH-VEG 8® or sold by Silab under the trademark Raffermine®); and plant hormones such as giberrellins and cytokinins.

The agents for stimulating keratinocyte proliferation that may be used in the composition according to the invention especially comprise retinoids such as retinol and its esters, including retinyl palmitate; extracts of nut cakes sold by Gattefosse; and extracts of Solanum tuberosum sold by Sederma.

The agents for stimulating keratinocyte differentiation comprise, for example, minerals such as calcium; the extract of lupin sold by Silab under the trademark Photopreventine®; sodium beta-sitosteryl sulphate sold by Seporga under the trademark Phytocohesine®; and the extract of corn sold by Solabia under the trademark Phytovityl®.

The term “tensioning agent” means a compound capable of exerting tension on the skin, the effect of which is to temporarily fade out irregularities on the skin's surface, such as wrinkles and fine lines.

Among the tensioning agents that may be used in the composition according to the present invention, mention may be made especially of:

(1) polyurethane latices or acrylic-silicone latices, in particular those described in patent application EP-1,038,519, such as a propylthio(polymethyl acrylate), propylthio(polymethyl methacrylate) or propylthio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are sold especially by 3M under the trademarks VS 80, VS 70 or L021,

(2) soybean or wheat plant proteins, and/or

(3) sodium magnesium silicates (Laponites).

Among the derivatives capable of promoting lipolysis that may be found are:

1) phosphodiesterase inhibitors such as:

xanthine derivatives, for instance caffeine and its derivatives, especially the 1-hydroxyalkylxanthines described in document FR-A-2,617,401, caffeine citrate, theophylline and its derivatives, theobromine, acefylline, aminophylline, chloroethyltheophylline, diprofylline, diniprophylline, etamiphylline and its derivatives, etofylline and proxyphylline;

combinations containing xanthine derivatives, for instance the combination of caffeine and silanol (caffeine methylsilanetriol derivative), and for example the product sold by Exsymol under the name caféisilane C;

compounds of natural origin containing xanthine bases, and especially caffeine, such as extracts of tea, of coffee, of guarana, of maté, of cola (Cola nitida) and especially the dry extract of guarana fruit (Paulina sorbilis) containing 8% to 10% caffeine;

ephedrine and its derivatives which may be found especially in natural form in plants such as Ma Huang (Ephedra plant);

2) plant extracts and extracts of marine origin, which are either active on the receptors to be inhibited, such as the β-2-blockers and the NPY-blockers (described in patent EP-838,217), or inhibit the synthesis of the LDL or VLDL receptors, or are active in stimulating the P receptors and the proteins G, leading to the activation of adenyl cyclase. Examples of plant extracts of this type that may be mentioned include:

Garcinia cambogia,

extracts of Bupleurum chinensis,

extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree,

extracts of ginkgo biloba,

extracts of horsetail,

extracts of escin,

extracts of cangzhu,

extracts of Chrysanthemum indicum,

extracts of dioscorea plants rich in diosgenin or pure diosgenin or hecogenin, and derivatives thereof,

extracts of plants of the genus Armeniacea, Atractylodis platicodon, Sinom-menum, Pharbitidis or Flemingia,

extracts of Coleus such as C. forskohlii, C. blumei, C. esquirolii, C. scutellaroides, C. xanthantus and C. barbatus, such as the extract of the root of Coleus barbatus containing 60% forskolin,

extracts of Ballota,

extracts of Guioa, of Davallia, of Terminalia, of Barringtonia, of Trema or Antirobia,

extracts of marine origin that may be mentioned include: extracts of algae or of phytoplankton, such as rhodysterol or the extract of Laminaria digitata sold under the name PHYCOX75 by Secma, the alga skeletonema described in patent FR-2,782,921 or the diatoms described in patent FR-2,774,292;

3) peptides or proteins:

the peptides derived from parathyroid hormone as described in patents FR-2,788,058 and FR-2,781,231 from Sederma or the peptides described in document FR-2,786,693, or alternatively any other peptide with lipolytic properties,

protamines and derivatives thereof such as those described in document FR-A-2,758,724.

The amount of lipolytic active agents may vary within a wide range and depends on the nature of the active agent(s) used. In general, the slimming active agent(s) is (are) present in a concentration ranging from 0.001% to 20% and preferably from 0.1% to 10% by weight relative to the total weight of the composition.

Active agents acting on the capillary circulation (vasoprotectors or vasodilators) that may be mentioned include flavonoids, ruscogenins, esculosides, escin extracted from common horse-chestnut, nicotinates, heperidine methyl chalcone, ruscus, essential oils of lavender or of rosemary, and extracts of Ammi visnaga.

The active agents that are preferred according to the present invention are extracts of corn and of soybean, extracts of the alga Macrocystis pyrifera, polysilicone-8, caffeine, extracts of ivy and Terminalia sericea. They are particularly suitable for compositions intended for redefining the contours of the face.

When the composition of the invention is an emulsion, the proportion of the fatty phase may range from 5% to 80% by weight and preferably from 5% to 50% by weight relative to the total weight of the composition. The oils, emulsifiers and co-emulsifiers used in the composition in emulsion form are chosen from those conventionally used in cosmetics or dermatology. The emulsifier, and optionally the co-emulsifier, are present in the composition in a proportion ranging from 0.3% to 30% by weight and preferably from 0.5% to 20% by weight relative to the total weight of the composition.

As oils that may be used in the invention, mention may be made of mineral oils (liquid petroleum jelly), plant oils (liquid fraction of karite butter), animal oils, synthetic oils (purcellin oil or hydrogenated polyisobutene), silicone oils and fluoro oils (perfluoropolyethers). Fatty alcohols, fatty acids and waxes may also be used as fatty substances.

As emulsifiers that may be used in the invention, examples that may be mentioned include fatty acid esters of polyols, such as fatty esters of sorbitol, for instance sorbitan tristearate sold under the name Span 65 by ICI, or fatty esters of glycerol such as glyceryl monostearate, or alternatively PEG esters such as PEG-40 stearate sold under the name Myrj 52 by ICI. They may also be silicone emulsifiers such as the cetyl dimethicone copolyol sold under the name Abil EM90 by Goldschmidt.

Another subject of the present invention relates to the use of ascorbic acid or an analogue thereof in the culture medium of a reconstructed skin to reinforce the cohesion of the dermo-epidermal junction of the said reconstructed skin.

This use applies to all skins reconstructed in vitro, in particular reconstructed skins whose:

epidermis is differentiated and comprises four main layers: basal layer, suprabasal layer, granulous layer and horny layer, and also the proteins associated with this differentiation; and

dermal compartment is alive, i.e., it comprises viable cells of fibroblast type.

It may be, for example, a reconstructed skin comprising an epidermis consisting of normal human keratinocytes that have differentiated to form an orthokeratotic horny layer laying on a dermal equivalent comprising fibroblasts that have contracted a collagen gel.

Specifically, the Applicant has observed that the addition of vitamin C to the culture medium of a reconstructed skin throughout the phase of emersion induces a very large increase in the number of fibroblasts in the lattice. This increase is associated with a greater production of certain proteins of the extracellular matrix, in particular in the sub-epidermal zone. The most spectacular results are found in this region of the dermo-epidermal junction, with a more perpendicular arrangement of the basal keratinocytes and an increased synthesis of major components of the basal membrane, for instance tenascin and collagen VII.

This use thus makes it possible to obtain a marked improvement of the model. The reconstructed skins obtained are more similar to normal human skin, and in particular the phenomena observed on the reconstructed skins are more predictive of the phenomena that will be observed in vivo.

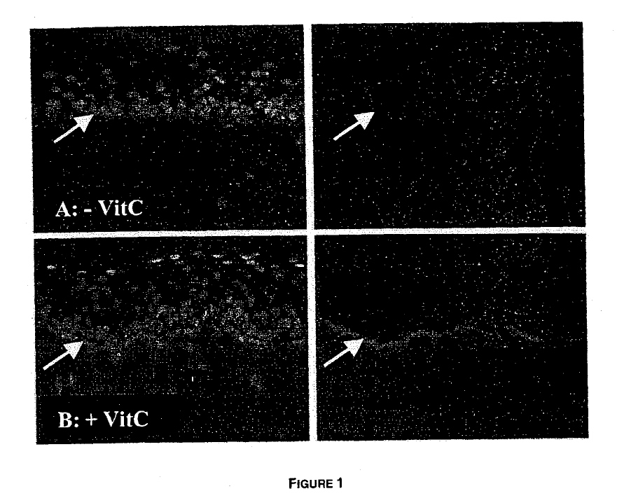

This emerges especially from the attached FIGS. 1 to 3, which are discussed in Example 3 below and in which:

FIG. 1 shows photographs of cross sections of reconstructed skin: for the images A, the reconstructed skin is grown in the absence of vitamin C and for the images B, the reconstructed skin is grown in the presence of vitamin C. For the left-hand images, the skin received two types of staining, one targeting the cell nuclei (spheroids at the top of the photographs), the other showing the presence of collagen VII (horizontal line). In the two right-hand images, only the staining showing the presence of collagen VII is represented.

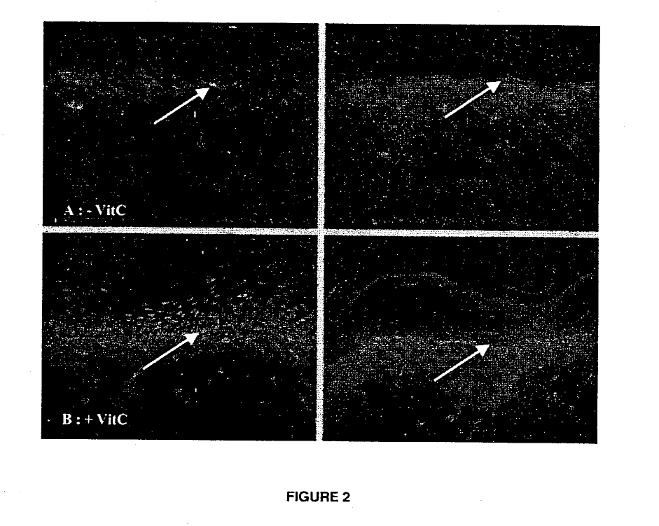

FIG. 2 represents photographs of cross sections of reconstructed skin: for the images A, the reconstructed skin is grown in the absence of vitamin C, and for the images B, the reconstructed skin is grown in the presence of vitamin C. For the left-hand images, the skin received two types of staining, one targeting the cell nuclei (spheroids at the top of the photographs), the other showing the presence of tenascin (horizontal line). In the two right-hand images, only the staining showing the presence of tenascin is represented.

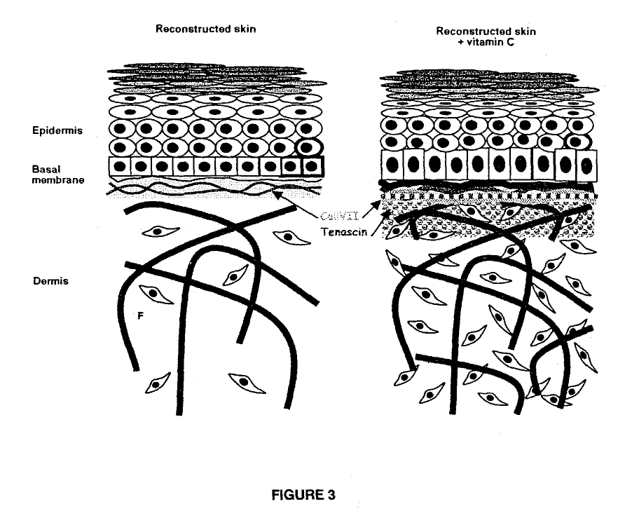

FIG. 3 is a diagrammatic representation of a cross section of reconstructed skin, the left-hand part of the diagram showing a reconstructed skin grown without vitamin C, and the right-hand part showing a reconstructed skin grown in the presence of vitamin C.

### EXAMPLES

The examples that follow illustrate the invention without in any way limiting its scope. The percentages are given on a weight basis relative to the total weight of the composition.

### Example 1

Gel containing vitamin C buffered to pH 4:
Gel:

| Phase A: | |
|---|---|
| Carrageenan | 1.5% |
| Lubrajel | 28.5% |
| Preserving agent | 0.2% |
| Deionized water qs | 95% |
| Sodium hydroxide | 0.5% |
| Phase B: | |
| Deionized water | 5.3% |
| Germall 115 | 0.3% |
| Dequest 2046 | 0.1% |

Solid:

| | |
|---|---|
| Ascorbic acid | 5% |

The gel is manufactured in a conventional manner by preparing, separately, by simple mixing of phases A and B at 70° C. and 40° C., respectively, followed by adding phase B to phase A with stirring at 40° C. and leaving the mixture to cool to room temperature with slow stirring.

The gel thus obtained is useful for redefining the contours of the face.

### Example 2

Water-in-oil emulsion:

| Oily phase: | |
|---|---|
| Dimethicone copolyol and cyclomethicone | 8% |
| (“3225C Formulation Aid” sold by Dow Corning) | |
| Phenyltrimethylsiloxytrisiloxane (“556 Fluid” | 15% |
| sold by Dow Corning) | |
| Tocopherol | 0.5% |
| Aqueous phase: | |
| Propylene glycol | 39.4% |
| Polyethylene glycol 400 | 13% |
| Disodium salt of ethylenediaminetetraacetic | 0.1% |
| acid (chelating agent) | |
| Ascorbic acid | 3% |
| Water | 21% |

The procedure for preparing the emulsion is as follows: the aqueous phase and the oily phase are prepared separately, and the aqueous phase is emulsified in the oily phase at room temperature with stirring using a homogenizer.

A white cream is obtained, which, when applied to the skin, makes it possible to accelerate cell renewal, to improve the surface aspect of the skin, and in particular to reduce the slackness of the skin.

### Example 3

Observation of the Effect of Adding Vitamin C on the Synthesis of Tenascin and Collagen VII:

The present example describes the effects of adding ascorbic acid on a reconstructed skin by microscopic observation of skin sections with immunohistochemical marking of the tenascin and collagen VII proteins.

1. Preparation of the Reconstructed Skin:

The reconstructed skin used is produced according to the protocol described in Asselineau et al. (Models in Dermato. Editions Loire and Maibach, 1987, Vol III, 1-7). The modifications to this protocol are:

the use of normal adult human dermal fibroblasts at a rate of 10⁶ cells per equivalent dermis;

the keratinocytes are inoculated at a rate of 50 000 cells per ring 1.5 cm in diameter. The keratinocytes used are obtained from the same donor and are at the 1st passage during the inoculation of the dermal equivalents;

the duration of the immersion phase is 7 days;

the duration of the emersion phase is 7 days.

2. Addition of Vitamin C:

The molecule used is magnesium ascorbyl phosphate (referred to hereinbelow as vitamin CPMg), used at a final concentration of 2.8′10⁻⁴ M.

The final change of medium of the immersion phase is performed in the presence of vitamin CPMg. The culture is then mounted on a grille for the emersion phase (7 days) and, during this phase, all the changes of medium (every 2 days) are performed in the presence of vitamin CPMg.

3.a. Analysis of the Collagen VII:

The reconstructed skins are analysed at the end of the emersion phase. A control sample (absence of vitamin CPMg) is systematically prepared and analysed in parallel.

The samples are taken and frozen in liquid nitrogen. The blocks are prepared in Tissue teck. The type VII collagen is detected by immunohistochemistry on frozen 5 μm sections. The standard technique of indirect immunofluorescence is performed with an anti-collagen VII monoclonal antibody (LH7.2, Chemicon International Inc., USA) and a fluorescein-coupled conjugate (FITC-conjugated Rabbit anti mouse inununoglobulins, DAKO, Denmark).

3.b. Analysis of the Tenascin:

The protocol used is that described in Point 3.a. above, the only difference being that in this case, the tenascin is detected with an anti-tenascin monoclonal antibody (TN2, Chemicon) and a fluorescein-coupled conjugate (FITC-conjugated Rabbit anti mouse immunoglobulins, DAKO, Denmark).

4. Observations:

4.a. Collagen VII:

By microscopic observation (see FIG. 1, images A: reconstructed skin grown in the absence of vitamin C, and images B: reconstructed skin grown in the presence of vitamin C), the presence of collagen VII is reflected by fluorescence in the junction zone between the epidermis and the dermis. It is found that the intensity and thickness of the zone of fluorescence, corresponding to the dermo-epidermal junction, is much greater in the sample to which vitamin C was added.

The left-hand and right-hand images in FIG. 1 correspond to the same zones of skin sections. In the left-hand image, all the cell nuclei have been stained with propidium iodide, allowing the tissue to be easily detected. The epidermis (very cellular and thus comprising many nuclei that may be visualized by pale circular marks above the line denoted by the arrow) is at the top of the section, and the dermis (much less cellular tissue comprising fibroblasts distributed in the extracellular matrix) is located at the bottom of the section. The zone of fluorescence revealing the presence of collagen VII is located at the junction between the epidermis and the dermis in the form of a linear marking (at the level of the arrow).

Measurement of the zone of fluorescence is performed by image analysis (Leica Q500W quantimeter) and normalized for the control and treated samples relative to an identical length of dermo-epidermal junction (arbitrary units).

| Collagen VII | Control | +Vitamin C |
|---|---|---|
| Fluorescence intensity/unit of length | 1.35 | 2.50 (×1.85) |

4.b. Tenascin:

The presence of tenascin is reflected by fluorescence in the junction zone between the epidermis and the dermis which diffuses into the superficial portion of the dermis. It is found that the intensity and thickness of the zone of fluorescence (see FIG. 2, images A: reconstructed skin grown in the absence of vitamin C, and images B: reconstructed skin grown in the presence of vitamin C), corresponding to the dermo-epidermal junction and to the superficial dermis, are much larger in the sample to which vitamin C was added.

The left-hand and right-hand images correspond to the same zones of skin sections. On the left-hand image, all the cell nuclei have been stained with propidium iodide, which allows the tissue to be easily detected. The epidermis (very cellular and thus comprising many nuclei that may be visualized by pale circular marks above the line denoted by the arrow) is at the top of the section, and the dermis (much less cellular tissue comprising fibroblasts distributed in the extracellular matrix) is at the bottom of the section. The tenascin revealed by fluorescent immunolabelling is distributed in the dermo-epidermal junction and in the sub-epidermal zone corresponding to the superficial dermis (at the level of the arrow).

Measurement of the zone of fluorescence is performed by image analysis (Leica Q500W quantimeter), and standardized for the control and treated samples relative to an identical length of dermo-epidermal junction (arbitrary units).

| Tenascin | Control | +Vitamin C |
|---|---|---|
| Fluorescence intensity/unit of length | 6.4 | 11.54 (×1.80) |

The results obtained thus show an improvement in the dermo-epidermal junction with vitamin CPMg, which is better represented diagrammatically in the attached FIG. 3.

Each patent, patent application and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for reinforcing the cohesion of the dermo-epidermal junction, comprising topically applying onto the skin of an individual subject in need of such treatment, a thus-effective amount of ascorbic acid or analogue thereof, formulated into a topically applicable, cosmetically/pharmaceutically acceptable medium therefor.

2. A regime or regimen for increasing the synthesis of tenascin, comprising topically applying onto the skin of an individual subject in need of such treatment, a thus-effective amount of ascorbic acid or analogue thereof, formulated into a topically applicable, cosmetically/pharmaceutically acceptable medium therefor.

3. A regime or regimen for increasing the synthesis of type VII collagen, comprising topically applying onto the skin of an individual subject in need of such treatment, a thus-effective amount of ascorbic acid or analogue thereof, formulated into a topically applicable, cosmetically/pharmaceutically acceptable medium therefor.

4. A regime or regimen for reinforcing the anchoring structures between the dermis and the epidermis, or improving the binding and adhesion of the epidermal cells to the matrix support, comprising topically applying onto the skin of an individual subject in need of such treatment, a thus-effective amount of ascorbic acid or analogue thereof, formulated into a topically applicable, cosmetically/pharmaceutically acceptable medium therefor.

5. A regime or regimen for improving the biomechanical properties of the skin, or increasing the resistance of the skin to mechanical challenges, comprising topically applying onto the skin of an individual subject in need of such treatment, a thus-effective amount of ascorbic acid or analogue thereof, formulated into a topically applicable, cosmetically/pharmaceutically acceptable medium therefor.

6. A regime or regimen for promoting skin repair or cicatrization, comprising topically applying onto the skin of an individual subject in need of such treatment, a thus-effective amount of ascorbic acid or analogue thereof, formulated into a topically applicable, cosmetically/pharmaceutically acceptable medium therefor.

7. A regime or regimen for improving the condition or growth of the hair, and/or for retarding or preventing hair loss, comprising topically applying onto the hair and/or scalp of an individual subject in need of such treatment, a thus-effective amount of ascorbic acid or analogue thereof, formulated into a topically applicable, cosmetically/pharmaceutically acceptable medium therefor.

8. The regime or regimen as defined by any of claims 1, 2 or 3, comprising topically applying a salt, ester or sugar of ascorbic acid.

9. The regime or regimen as defined by claim 8, comprising topically applying sodium ascorbate, magnesium ascorbyl phosphate, sodium ascorbyl phosphate, an acetic, propionic or palmitic ester of ascorbic acid, glycosyl ascorbic acid, or mixture thereof.

10. The regime or regimen as defined by any of claims 1, 2 or 3, the formulation comprising from 0.001% to 20% by weight of said ascorbic acid or analogue thereof.

11. The regime or regimen as defined by claim 10, the formulation comprising from 0.1% to 15% by weight of said ascorbic acid or analogue thereof.

12. The regime or regimen as defined by claim 11, the formulation comprising from 1% to 10% by weight of said ascorbic acid or analogue thereof.

13. The regime or regimen as defined by any of claims 1, 2 or 3, the formulation also comprising at least one hydrophilic or lipophilic gelling agent, anti-glycation agent, agent for stimulating the synthesis of dermal or epidermal macromolecules and/or for preventing their degradation, agent for stimulating the proliferation of fibroblasts or keratinocytes and/or for differentiating keratinocytes, tensioning agent, lipolytic active agent or active agent that has direct or indirect activity on reducing adipose tissue, agent that acts on capillary circulation, hydrophilic or lipophilic active agent, preservative, antioxidant, solvent, fragrance, filler, screening agent, pigment, odor absorber, dyestuff or mixture thereof.

14. The regime or regimen as defined by any of claims 1, 2 or 3, the formulation comprising an aqueous, aqueous-alcoholic or oily solution, dispersion of the lotion or serum type, an aqueous, anhydrous or oily gel, emulsion of liquid or semi-liquid consistency of the milk type, obtained by dispersing a fatty phase in an aqueous phase (O/W) or conversely (W/O), or a triple emulsion (W/O/W or O/W/O), suspension or emulsion of soft, semi-solid or solid consistency of the cream or gel type, microemulsion, microcapsules, microparticles or a vesicular dispersion of ionic and/or nonionic type.

15. A method for reinforcing the cohesion of the dermo-epidermal junction of a reconstructed skin, comprising introducing into the culture medium therefor, a thus-effective amount of ascorbic acid or analogue thereof.
